SEQUENCE LISTING (11 sequences)

SEQ ID NO: 1 - DNA, 30 bp, Artificial Sequence
  Description of Artificial SequencePCR Primer
gaggtataaaacccatgggtactgcactcg

SEQ ID NO: 2 - DNA, 27 bp, Artificial Sequence
  Description of Artificial SequencePCR Primer
tatcgtgaggaagcttcatcgccattg

SEQ ID NO: 3 - DNA, 28 bp, Artificial Sequence
  Description of Artificial SequencePCR Primer
gaaggtggcaccatggcaccggacatta

SEQ ID NO: 4 - DNA, 27 bp, Artificial Sequence
  Description of Artificial SequencePCR Primer
ggataatgtccgaattcatgctgccac

SEQ ID NO: 5 - DNA, 30 bp, Artificial Sequence
  Description of Artificial SequencePCR Primer
gacgcttataaaccatggatattaatactg

SEQ ID NO: 6 - DNA, 27 bp, Artificial Sequence
  Description of Artificial SequencePCR Primer
gcgctgaggtctagatcgtgaagaagg

SEQ ID NO: 7 - DNA, 28 bp, Artificial Sequence
  Description of Artificial SequencePCR Primer
acacaggaaacatctagaaccatgatta

SEQ ID NO: 8 - DNA, 83 bp, Artificial Sequence
  Description of Artificial SequencePCR Primer
cctggcaaccagcgccgacaaaggcgcggtgcgcgataaatcgaaactggggggttaatagccgccgtcccgtcaagtcagcg

SEQ ID NO: 9 - DNA, 82 bp, Artificial Sequence
  Description of Artificial SequencePCR Primer
cacaaatgacgttgtcgcgcgggtaggcctgataagcgaagcgctatcaggcatttttccccccggatccgtcgacctgcag

SEQ ID NO: 10 - DNA, 25 bp, Artificial Sequence
  Description of Artificial SequencePCR Primer
gaaaaatcgtgaacgtcaggtctcc

SEQ ID NO: 11 - DNA, 20 bp, Artificial Sequence
  Description of Artificial SequencePCR Primer
attcctgtttgtaattgtcc

(19) United States
(12) Patent Application Publication
(10) Pub. No.: US 2004/0209367 A1
(43) Pub. Date: Oct. 21, 2004

(54) Mutagenesis technique

(76) Inventors: Ian Charles, London (GB); Kerry Wheeler, London (GB)

Correspondence Address: FULBRIGHT & JAWORSKI, LLP, 1301 MCKINNEY, SUITE 5100, HOUSTON, TX, 77010-3095, US

(21) Appl. No.: 10/472,094

(22) PCT Filed: Mar. 18, 2002
(86) PCT No.: PCT/GB02/01274

(30) Foreign Application Priority Data
May 16, 2001 (GB) 00106567.1

(51) Int. Cl. C12N 15/74; C12N 1/21

(52) U.S. Cl. 435/471; 435/252.3

(57) ABSTRACT

A method for replacing a target nucleotide/polynucleotide sequence of a bacterial chromosome with a different nucleotide/polynucleotide sequence, which method comprises: (a) providing a bacterium which is capable of expressing: (1) the γ genes exo or a functional equivalent thereof and bet or a functional equivalent thereof, but not the γ gene gam or a functional equivalent thereof; or (2) the γ gene bet or a functional equivalent thereof and gam or a functional equivalent thereof; (b) providing a polynucleotide construct which comprises: (i) a sequence which corresponds to a first sequence flanking the left hand side of the target sequence; (ii) a second sequence corresponding to a sequence flanking the right hand side of the target sequence; and (iii) positioned between (i) and (ii), the donor sequence; and (c) introducing the polynucleotide construct into the bacterium, thereby to replace the target nucleotide sequence with the sequence different from that of the target nucleotide sequences. The method can be used in the preparation of attenuated bacteria and in the identification of essential genes.

### FIELD OF THE INVENTION

This invention relates to a method for carrying out allelic exchange and to vectors for use in that method.

### BACKGROUND TO THE INVENTION

Over the last few years, the genomic sequences of a large number of bacteria have been reported. This flood of data has resulted in the identification of thousands of DNA sequences that currently have no functions ascribed to them. A challenge for functional genomics strategies is to characterise these unknown genes. The first step in any process of functional analysis often involves the deletion of the chromosomal copy of the target gene to generate a null-mutant. Mutant strains generated in this way can subsequently be analysed for functional changes compared to the phenotype of a wild-type parent strain. Typically, this process can be carried out in a variety of microorganisms by the process of allelic exchange.

For many microorganisms, the process of allelic exchange is complex and involves the use of suicide plasmids, large fragments of cloned target DNA flanking an antibiotic selectable marker and a counter-selection process. For example, construction of an allelic replacement mutant in Escherichia coli and Salmonella typhirninum typically involves the identification of left- and right- arms of a target gene. These fragments are cloned flanking an antibiotic selection marker (such as kanamycin, kan) into a plasmid suicide replicon. The origin of replication for this plasmid is selected so that replication cannot occur in the target strain where the allelic exchange will take place. When antibiotic selection is applied under these conditions, resistant colonies arise from chromosomal integration of the antibiotic resistance gene. Allelic exchange is mediated by recombination between the left- and right- arms of the target gene on the plasmid and the copy of the corresponding gene on the chromosome. Often, however, this recombination event produces a single cross-over event, integrating the entire plasmid in the host genome and resulting in the presence of tandem copies of the target gene. This duplication can be resolved by a second homologous recombination event that leaves a single copy in the genome—either the wild-type copy, or the mutant copy disrupted by the kanamycin resistance determinant. In order to select for this second recombination event, allelic exchange vectors often incorporate a counter-selectable marker, such as the sacB gene from Bacillus subtilis.

Candidate mutants are isolated following selective plating on antibiotic containing medium. The identity of the mutation must be confirmed by several steps including: (i) Southern blotting; (ii) PCR cloning of the disrupted junction; and (iii) DNA sequencing. Overall, the process to generate mutants in Gram negative bacteria like E. coli and S. typhimurium is complex and time consuming.

By comparison, the yeast Saccharomyces cerevisiae, possesses a highly efficient homologous recombination system and this has allowed the development of high through-put allelic exchange procedures to generate large numbers of mutants. For example, the length of the flanking DNA regions used to direct homologous recombination in yeast need not be the kilo-based sized fragments required in Salmonella spp. and can be reduced in length down to 30-45 bp. These short fragments can be readily generated by synthetic oligonucleotides allowing the construction of gene-disruption cassettes that can be produced by Polymerase Chain Reaction (PCR) without the need for cloning steps (Guldener et al., 1996, Nucleic Acids Res. 24, 2519-2524). Furthermore, no suicide replicons are required, as the PCR fragment can be directly introduced in to yeast to direct recombination.

Unfortunately, this system is not applicable to wild-type E. coli because of the presence of intracellular exonucleases that rapidly degrade linear DNA (Lorenz and Wackemagel, 1994, Microbiol. Rev. 58, 563-602). Recently several publications have reported that these limitations may be overcome for the generation of PCR-driven allelic exchange mutants in E. coli. Two basic systems have been developed, with recombination mediated either by the E. coli recE, recT proteins, so-called ET cloning (Zhang et al., 1998, Nat. Genetn. 20, 123-128; Muyrers et al., 1999, Nucleic Acids Res. 27, 1555-1557) or the λ genes, exo, bet and gam (Datsenko et al., 2000, Proc. Natl. Acad. Sci. USA 97, 6640-6645; Yu et al., 2000, Proc. Natl. Acad. Sci. USA 97, 5978-5983).

We have now developed a new method for carrying out allelic exchange in bacteria.

### SUMMARY OF THE INVENTION

Previously reported methods for carrying out allelic exchange in E. coli that make use of λ genes have all asserted that the presence of all three of exo, bet and gam is required. We have now shown, surprisingly, that this is not correct. In fact allelic exchange can be carried out in the presence of: (i) only exo and bet; or (ii) only bet.

According to the invention there is thus provided a method for replacing a target sequence of a bacterial chromosome with a donor sequence, which method comprises:

(a) providing a bacterium which is capable of expressing:

(1) the λ genes exo or a functional equivalent thereof and bet or a functional equivalent thereof, but not the λ gene gam or a functional equivalent thereof; or

(2) the λ gene bet or a functional equivalent thereof, but not the λ genes exo or a functional equivalent thereof and gam or a functional equivalent thereof;

(b) providing a polynucleotide construct which comprises:

(i) a sequence which corresponds to a first sequence flanking the left hand side of the target sequence;

(ii) a second sequence corresponding to a sequence flanking the right hand side of the target sequence; and

(iii) positioned between (i) ,and (ii), the donor sequence; and

(c) introducing the polynucleotide construct into the bacterium, thereby to replace the target nucleotide sequence with the sequence different from that of the target nucleotide sequence.

The invention also provides:

a method of the invention for the preparation 6f a modified bacterium;

a method of the invention for the preparation of an attenuated bacterium, wherein the target sequence comprises all or part of a gene which is required for pathogenicity;

an attenuated bacterium obtained by a method of the invention for the preparation of an attenuated bacterium;

an attenuated bacterium which is capable of expressing:

(a) the λ genes exo or a functional equivalent thereof and bet or a functional equivalent thereof, but not the λ gene gam or a functional equivalent thereof; or

(b) the λ gene bet or a functional equivalent thereof but not the λ genes exo or a functional equivalent thereof and gam or a functional equivalent thereof, and which is attenuated by a non-reverting mutation in at least one gene which is required for pathogenicity;

a vaccine comprising an attenuated bacterium of the invention and a pharmaceutically acceptable carrier or diluent;

an attenuated bacterium of the invention for use in a method for vaccinating a human or animal;

use of a bacterium of the invention in the manufacture of a medicament for vaccinating a human or animal;

a method for raising an immune response in a mammalian host, which comprises administering to the host a bacterium or a vaccine composition of the invention;

a method of the invention for identifying an essential gene wherein inability to replace the target sequence is indicative of that target sequence comprising all or part of an essential gene;

use of an essential gene identified by a method of the invention for identifying an essential gene, or the polypeptide encoded by a said gene, in a method for identifying an inhibitor of transcription and/or translation of that gene and/or activity of a polypeptide encoded by that gene;

a method for identifying:

(i) an inhibitor of transcription and/or translation of an essential gene identified by a method of the invention for identifying an essential gene; and/or

(ii) an inhibitor of activity of a polypeptide encoded by a said gene, which method comprises determining whether a test substance can inhibit transcription and/or translation of a said gene and/or activity of a polypeptide encoded by a said gene;

a method for identifying: (i) an inhibitor of transcription and/or translation of an essential gene; and/or (ii) an inhibitor of activity of a polypeptide encoded by a said gene, which method comprises:

(a) identifying an essential gene by a method of the invention for identifying an essential gene; and

(b) determining whether a test substance can inhibit transcription and/or translation of a said gene and/or activity of a polypeptide encoded by a said gene;

an inhibitor identified by a method as set out above;

an inhibitor of the invention for use in a method of treatment of the human or animal body by therapy;

use of an inhibitor of the invention in the manufacture of a medicament for use in the treatment of a bacterial infection;

a pharmaceutical composition comprising an inhibitor of the invention and a pharmaceutically acceptable carrier or diluent;

a method of treating a host suffering from a bacterial infection, which comprises administering to the host an effective amount of an inhibitor of the invention;

a method for the preparation of a pharmaceutical composition, which method comprises:

(a) identifying: (i) an inhibitor of transcription and/or translation of an essential gene; and/or (ii) an inhibitor of activity of a polypeptide encoded by a said gene by a method as set out above; and

(b) formulating the inhibitor identified in step (a) with a pharmaceutically acceptable carrier or diluent; and

a method of treating a host suffering from a bacterial infection, which method comprises:

(a) identifying: (i) an inhibitor of transcription and/or translation of an essential gene; and/or (ii) an inhibitor of activity of a polypeptide encoded by a said gene by a method as set out above;

(b) formulating the inhibitor identified in step (a) with a pharmaceutically acceptable carrier or diluent; and

(c) administering to the host an effective amount of the pharmaceutical composition of step (b).

### BRIEF DESCRIPTION OF THE DRAWING

FIG. 1 sets out a schematic representation of one particular embodiment of allelic replacement technique of the invention.

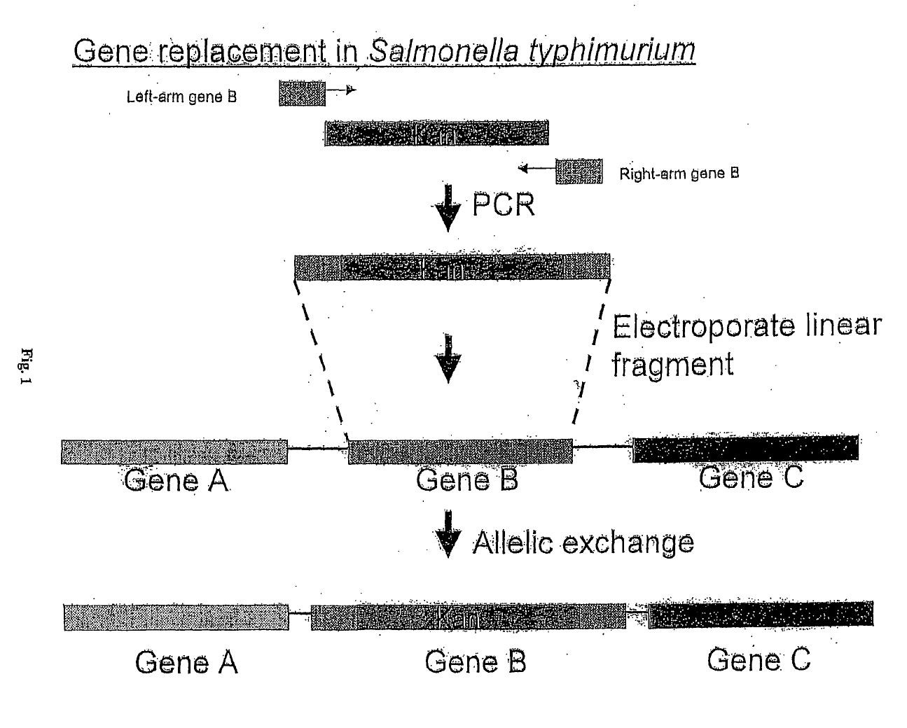

### BRIEF DESCRIPTION OF THE SEQUENCE LISTING

SEQ ID NO: 1 sets out the sequence of the bet forward primer.

SEQ ID NO: 2 sets out the sequence of the exo reverse primer.

SEQ ID NO: 3 sets out the sequence of the exo forward primer.

SEQ ID NO: 4 sets out the sequence of the bet reverse primer.

SEQ ID NO: 5 sets out the sequence of the gain forward primer.

SEQ ID NOS: 6 and 7 set out the sequences of the primers used to amplify the kan-resistance determinant from pUC4K.

SEQ ID NO: 8 sets out the sequence of the ilvA forward primer.

SEQ ID NO: 9 sets out the sequence of the ilvA reverse primer.

SEQ ID NO: 10 sets out the sequence of the ilvA test primer.

SEQ ID NO: 11 sets out the sequence of the universal kan test primer.

### DETAILED DESCRIPTION OF THE INVENTION

The invention provides a general method for replacing a specific genomic sequence in a bacterium with any other desired sequence. The sequence to be replaced and the sequence with which it is replaced can be conveniently referred to using the terms “target sequence” and “donor sequence” respectively.

The method of the invention provides a simple and fast way of analysing the bacterial genome. The method allows null mutants to be created, for example, and thus provides an important tool in the analysis of genes for which no function is known.

If a particular sequence, for example an open reading frame or a part thereof or a region 5′ to an open reading frame (for example a promoter) or part thereof, cannot be replaced, that may indicate that the sequence is essential for viability in the bacterium in which the sequence occurs, i.e. that the sequence represents all or part of an essential gene. Esseritial sequences are targets for the development of new antibiotics. Thus, the invention provides a method for generating new targets for screening for new antimicrobial substances.

In addition, genes of known function may be replaced. That is, modified bacteria may be prepared by use of the allelic exchange method of the invention. Therefore, the invention provides a method for the preparation of a modified bacterium.

This is important if, for example, mutation of a particular gene is known to result in attenuation of a pathogenic bacterium. Thus, the invention provides a method for the preparation of an attenuated bacterium. Such attenuated bacteria may be used in the preparation of vaccine compositions and in the vaccination of humans or animals.

The invention further provides a bacterium which is capable of expressing: (a) the λ genes exo or a functional equivalent thereof and bet or a functional equivalent thereof, but not the λ gene gam or a functional equivalent thereof; or (b) and which carries a mutation in one or more of its genes. The bacterium may, for example carry a non-reverting mutation in at least one gene, for example in two, three, four or five genes. Typically, at least one of those non-reverting mutations is in a gene which is required for pathogenicity.

In a method of the invention the bacterium, which is to have a specific part of its genomic sequence replaced (the target sequence), is provided in a form such that it expresses the λ genes exo or a functional equivalent thereof and bet or a functional equivalent thereof. Such a bacterium does not, however, express the λ gene gam or a functional equivalent thereof. Alternatively, the bacterium is provided in a form such that it expresses the λ gene bet or a functional equivalent thereof. Such a bacterium does not, however, express the λ genes gam or a functional variant thereof or exo or a functional variant thereof.

A polynucleotide construct is then provided which comprises sequences corresponding to the bacterial sequences that flank both sides of the target genomic sequence separated by the sequence which is to replace the target sequence (the donor sequence). That is, the construct comprises a first sequence which corresponds to the bacterial sequence flanking the left hand side of the target sequence, a second sequence which corresponds to the bacterial sequence flanking the right hand side of the target sequence and the donor sequence is positioned between those two sequences. The donor sequence is typically different from the target sequence.

The polynucleotide construct is then introduced into the bacterium by any suitable means. Homologous recombination between the bacterial genomic sequences flanking the target sequence and the sequences of the construct which correspond to those flanking sequences leads to replacement of the target sequence with the donor sequence.

Successful recombinants can be selected for by determining whether the bacterium has incorporated the target. If recombinants cannot be detected, this is a strong indication that the target sequence is essential for viability of the bacterium. That is, the target sequence may comprise all or part of an essential gene, for example the target sequence may comprise all or part of a control sequence, such as a promoter, or may comprise or all or part of a coding sequence.

The sequence to be replaced in a bacterium, the target sequence, may be any sequence. Typically, the target sequence will be a coding sequence, for example consisting of an entire open reading frame or a fragment thereof. However, the target sequence may consist entirely of non-coding sequence, for example regions 5′ to the coding sequence of a gene, or comprise both coding and non-coding sequences.

The target sequence may be of any suitable size, for example from 1 bp to 50kb in length. Typically, however, the minimum size of a target sequence will be at least 10 bp, at least 25 bp, at least 50 bp or more preferably at least 100 bp or typically at least 500 bp in length. In general the maximum length of a target sequence will be up to 30 kbp, up to 15 kbp, up to 5 kbp or up to 2 kbp. The length of the target sequence may preferably be up to 1 kb and typically up to 800 bp. Any combination of the above mentioned lower and upper lengths may be used to defined a target sequence of the invention.

The bacterium to be used in a method of the invention is capable of expressing the λ genes exo or a functional equivalent thereof and bet or a functional equivalent thereof. Alternatively, the bacterium is capable of expressing the λ gene bet or a functional equivalent thereof The λ genes exo and bet are well known to those skilled in the art and the coding sequences of those genes have the following GenBank accession numbers:

| | | |
|---|---|---|
| exo | Accession no: NP_040616 | PID: g9626280. |
| bet | Accession no: NP_040617 | PID: g9626281. |

A functional equivalent of exo or bet is a coding sequence which codes for a polypeptide which can provide a similar enzymatic activity to the polypeptides encoded by the coding sequences having the GenBank accession numbers given above. A polypeptide encoded by a functional equivalent of exo or bet has a similar enzymatic activity in the sense that it catalyses the same reaction with substantially the same specificity as exo or bet and catalyses that reaction at a rate of at least 60%, at least 70%, preferably at least 80%, generally at least 90%, for example at least 95%, typically at least 99% and most preferably at a rate substantially the same as that of the exo or bet polypeptide when measured under the same conditions.

Functional equivalents of exo or bet may be obtained by any method known to those skilled in the art. For example, they can be generated by nucleotide substitutions of the wild type sequence of exo or bet, for example from 1, 2 or 3 to 10, 25, 50 or 100 substitutions. Wild type sequences may alternatively or additionally be modified by one or more insertions and/or deletions and/or by an extension at either or both ends to give functional equivalents. The modified polynucleotide typically encodes a polypeptide which has activity as defined above. Degenerate substitutions may be made and/or substitutions may be made which would result in a conservative amino acid substitution when the modified sequence is translated.

Typically a functional equivalent will share at least 70% identity, at least 80% identity, at least 90% identity, at least 95% identity, or at least 99% identity with the wild type exo or bet sequence over at least 20, preferably at least 30, for instance at least 40, at least 60, or more preferably at least 100 contiguous nucleotides or most preferably over the full length of the wild type exo or bet sequence.

A functional equivalent may also be capable of hybridising under selective conditions to the complement of the wild type exo or bet sequence. A sequence which can hybridise to the complement of its corresponding sequence can typically hybridise to that coding sequence at a level significantly above background. Background hybridization may occur, for example, because of other nucleotide sequences used in a hybridisation reaction. The signal level generated by the interaction between a functional equivalent and the complement of its corresponding sequence is typically at least 10 fold, preferably at least 100 fold, as intense as interactions between other sequences and the exo or bet coding sequences. The intensity of interaction may be measured, for example, by radiolabelling the probe, e.g. with ³²P.

Selective hybridisation may typically be achieved using conditions of medium to high stringency (for example, 2×SSC [0.15M sodium chloride and 0.015M sodium citrate] at about 50° C. to about 60° C.). However, such hybridisation may be carried out under any suitable conditions known in the art (see Sambrook et al (1989) Molecular Cloning: A Laboratory Manual). For example, if high stringency is required suitable conditions include from 0.1 to 0.2×SSC at about 60° C. to about 65° C. If lower stringency is required suitable conditions include 2×SSC at 60° C.

A bacterium for use in the allelic exchange method of the invention does not comprise the λ gene gam or a functional equivalent thereof and thus is not capable of expressing a polypeptide encoded by gam or a polypeptide having similar enzymatic activity thereto.

The exo and/or bet genes (or functional equivalents thereof) may not occur naturally in a bacterial species selected for use in the invention and therefore exo or bet or functional equivalents of either thereof may need to be provided from a heterologous source. That is, a cell for use in a method of the invention may be a recombinant cell. Thus, an exo and a bet gene or a bet gene alone or functional equivalents of any thereof may be transferred into a bacterium; typically so that the transferred gene(s) is/are capable of being expressed. The expression of a transferred exo and/or bet genes or functional equivalents of either thereof may be stable or may be transient. The expression may be constitutive or may only occur in the presence of a particular stimulus, for example in the presence of a particular compound. Techniques for transferring genes into bacterial cells are well known to those skilled in the art.

The exo and/or bet genes or functional equivalents of either thereof may comprise part of a plasmid and be capable of being expressed from that plasmid when it is transferred into a bacterial cell. Alternatively, the exo and/or bet genes or functional equivalents of either thereof may comprise part of a prophage and be capable of being expressed from that prophage when it is transferred into a bacterial cell.

Expression of the exo and/or bet genes or functional equivalents of either thereof may be driven in a bacterium by any promoter which can drive expression in that bacterium. If a plasmid is used to transfer the exo and bet genes or a bet gene alone or functional equivalents of any thereof into a bacterium, it may be convenient to use constitutive promoters such as the tac or trc promoters. Altematively, it may be convenient to use a promoter which drives expression in the presence of a particular compound (an inducer), at a particular temperature, or under the control of some other regulatory process, for example the T7 promoter systems.

Thus, a promoter such as an arabinose inducible promoter may be used, for example the pBAD promoter. Regulated promoters may be preferred, because the exo and bet genes or a bet gene alone or functional derivatives of any thereof can then be expressed only when homologous recombination is required. This may help to reduce any unwanted recombination events mediated by the exo and bet or bet alone polypeptides or polypeptides similar thereto.

If a prophage is used to express exo and bet or bet alone or functional derivatives of any thereof, it may be convenient to engineer controlled expression using the λ cI-repressor. Use of this system allows expression of exo and bet or bet alone to be controlled by temperature. Growth of cells containing the prophage at 32° C. results in no expression of exo and bet or bet alone, whereas growth of cells containing the prophage at 42° C. results in expression of exo and bet or bet alone. Use of such a system may require cells containing the prophage to be grown to be at the permissive temperature for a short time, for example from 2 to 30 minutes, for example from 5 to 15 minutes, before transfer of the construct into the cells.

The construct for use in the allelic exchange technique of the invention comprises:

(a) a sequence which corresponds to a first sequence flanking the left hand side of the target nucleotide/polynucleotide sequence;

(b) a second sequence corresponding to a sequence flanking the right hand side of the target nucleotide/polynucleotide sequence; and

(c) positioned between (a) and (b), the sequence which will replace the target nucleotide sequence.

The construct of the invention is used in a linear form for transfer into the bacterium to be studied. The construct may be, for example, a double-stranded DNA (dsDNA) construct or a single-stranded DNA (ssDNA) construct. The latter type of construct, ssDNA constructs, are preferred when the bacterium used in the method of the invention is capable of expressing bet or a functional derivative thereof alone.

Sequences (a) and (b) correspond to sequences which flank the target sequence in the bacterium. Such sequences have to be sufficiently similar to the corresponding sequence in the bacterium and of sufficient length for homologous recombination to occur between the construct and the bacterial chromosome. Typically, sequences (a) and (b) will be sufficiently dissimilar such that insertion of the target sequence can only occur in one orientation.

Sequences (a) and (b) do not need to be of the same length. In general, sequences (a) and (b) may be, independently, at least 10 bp in length, at least 20 bp, at least 50 bp in length, at least 60 bp in length, at least 75 bp in length or at least 100 bp in length. Typically, sequences (a) and (b) will be, independently, up to 200 bp in length, up to 300 bp in length, up to 500 bp in length, up to 750 bp in length, up to 1 kbp in length or up to 2 kbp in length.

Typically, a sequence (a) or (b) for use in the invention will share 100% identity over their entire length With the sequence to which they correspond on the bacterial chromosome. However, a sequence (a) or (b) suitable for use in the invention may share, independently, at least 60% identity, at least 70% identity, at least 80% identity, at least 90% identity, at least 95% identity, or at least 99% identity with the corresponding bacterial sequence over a contiguous stretch of nucleotides representing at least 50% of its length, at least 60% of its length, at least 70% of its length, at least 80% of its length, at least 90% of its length, at least 95% of its length or at least 99% its length. Any combination of the above mentioned percentage identity and percentage length may be used to define a sequence (a) or (b) suitable for use in the invention, with greater % identity to the corresponding sequence on the bacterial chromosome over a greater percentage of the length of the sequence being preferred.

Sequence identities referred to in this specification can be calculated using techniques well known to those skilled in the art. For example, the UWGCG Package provides the BESTFIT program which can be used to calculate homology (for example used on its default settings) (levereux et al (1984) Nucleic Acids Research 12, p387-395). The PILEUP and BLAST algorithms can be used to calculate homology or line up sequences (typically on their default settings), for example as described in Altschul S. F. (1993) J Mol Evol 36:290-300; Altschul, S, F et al (1990) J Mol Biol 215:403-10.

Software for performing BLAST analyses is publicly available through the National Centre for Biotechnology Information (http://www.ncbi.nlm.nih.gov/). This algorithm involves first identifying high scoring sequence pair (HSPs) by identifying short words of length W in the query sequence that either match or satisfy some positive-valued threshold score T when aligned with a word of the same length in a database sequence. T is referred to as the neighbourhood word score threshold (Altschul et al, supra). These initial neighbourhood word hits act as seeds for initiating searches to find HSPs containing them. The word hits are extended in both directions along each sequence for as far as the cumulative alignment score can be increased. Extensions for the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program uses as defaults a word length (W) of 11, the BLOSUM62 scoring matrix (see Henikoff and Henikoff (1992) Proc. Natl. Acad. Sci. USA 89: 10915-10919) alignments (B) of 50, expectation (E) of 10, M=5, N=4, and a comparison of both strands.

The BLAST algorithm performs a statistical analysis of the similarity between two sequences; see e.g., Karlin and Altschul (1993) Proc. Natl. Acad. Sci. USA 90: 5873-5787. One measure of similarity provided by the BLAST algorithm is the smallest sum probability (P(N)), which,provides an indication of the probability by which a match between two nucleotide sequences would occur by chance. For example, a sequence is considered similar to another sequence if the smallest sum probability in comparison of the first sequence to the second sequence is less than about 1, preferably less than about 0.1, more preferably less than about 0.01, and most preferably less than about 0.001.

A sequence suitable for use as a sequence (a) or (b) may also be capable of hybridising under selective conditions to the complement of the sequence to which it corresponds on the bacterial chromosome.

A sequence (a) or (b) which can hybridise to the complement of its corresponding sequence can typically hybridise to that coding sequence at a level significantly above background. Background hybridisation may occur, for example, because of other nucleotide sequences present in the bacterial genome. The signal level generated by the interaction between a polynucleotide of (a) or (b) and the complement of its corresponding sequence is typically at least 10 fold, preferably at least 100 fold, as intense as interactions between other sequences of the bacterial genome under investigation and the complement of the sequence in the bacterial genome corresponding to (a) or (b). The intensity of interaction may be measured, for example, by radiolabelling the probe, e.g. with ³²P.

Selective hybridisation may typically be achieved using conditions of medium to high stringency (for example, 2×SSC [0.15M sodium chloride and 0.015M sodium citrate] at about 50° C. to about 60° C.). However, such hybridisation may be carried out under any suitable conditions known in the art (see Sambrook et al (1989) Molecular Cloning: A Laboratory Manual). For example, if high stringency is required suitable conditions include from 0.1 to 0.2×SSC at about 60° C. to about 65° C. If lower stringency is required suitable conditions include 2×SSC at 60° C.

The donor sequence (c) may be any sequence with which it is desired to replace the target sequence. The donor sequence does not need to be the same length as the target sequence and can be shorter or longer than the target sequence. Typically, however, the donor and target sequence will be similar in length, for example, the donor sequence may be 50%, 60%, 70%, 80%, 90%, 95% or substantially the same length of the target sequence or vice versa.

It will be convenient for the donor sequence (c) to comprise a coding sequence which codes for a polypeptide which is readily detectable, so that bacteria in which homologous recombination has occurred can be easily identified. If the donor sequence (c) comprises a coding sequence, it will also typically comprise a promoter operably linked to that coding sequence. The promoter should be selected so that expression of the coding sequence will be driven in the bacterium into which the construct is transferred.

Suitable coding sequences are those coding for polypeptides which confer antibiotic resistance on bacteria in which they are expressed. For example, such a polypeptide could confer resistance to kanamycin, ampicillin or tetracycline. Alternatively, the coding sequence may encode a so-called reporter polypeptide. Such a polypeptide may be, for example, a fluorescent or a colorimetric polypeptide. Such polypeptides are easy to detect using techniques will known to those skilled in the art.

Constructs for use in the invention can be prepared using, for example, recombinant DNA technology well known to those skilled in the art. Typically, it will be convenient to prepare constructs using Polymerase chain reaction (PCR). Thus primers may be designed which comprise 5′ sequence which provides the desired (a) or (b) sequence and which have 3′ sequences suitable for amplification of the donor sequence. More complex construction techniques may be required if the sequence is beyond the limits of the length of a PCR primer (a) or (b).

The primers described above may be used in PCR on a template which comprises the donor sequence to generate a linear construct suitable for use in the invention.

PCR can be carried out on, for example, a plasmid which contains the donor sequence and the linear PCR product obtained can then be purified from the PCR reaction mixture. It may, however, be more convenient to isolate the donor sequence as a linear fragment and to carry out PCR on that fragment. Use of this latter technique is advantageous over a technique which carries out PCR directly on a plasmid, because no purification of the construct is required after PCR. In order to ensure that the plasmid does not interfere with the subsequent transfer and selection (if selection is used), the resulting PCR reaction has to undergo a rigorous purification scheme involving: (1) gel purification; (2) digestion with a restriction endonuclease; and (3) a further round of gel purification. These steps have considerable cost and time implications if the allelic exchange method of the invention is to be applied to an entire genome analysis. Such steps are not required if PCR is carried out on a linear template.

As was mentioned above, ssDNA constructs are preferred for use with bacteria which are capable of expressing bet or a functional derivative thereof alone. Suitable ssDNA constructs can be generated by any technique. Such techniques are well known to those skilled in the art and include, for example, heat denaturation. Thus, a linear construct as described above may be subjected to heat denaturation, for example by heating to about 94° C. for about 1, 2 or 3 minutes followed by subsequent quenching on ice for about 1, 2 or 3 minutes. The resulting ssDNA constructs can be used as described below.

A construct, for example a dsDNA or a ssDNA construct, for use in the invention is transferred into a bacterium which is capable of expressing the λ genes exo and bet or a functional equivalent of either or both thereof, but which is not capable of expressing the λ gene gam or a functional equivalent thereof. Alternatively the construct is transferred into a bacterium which is capable of expressing the λ gene bet or a functional equivalent thereof, but which is not capable of expressing the λ genes exo and gam or a functional equivalent of either of both thereof The transfer may be carried out by any method. Suitable methods are will known to those skilled in the art, for example electroporation or thermal shock. Electroporation is preferred in the allelic exchange technique of the invention.

Recombinants, i.e. bacteria which have incorporated the donor sequence in place of the target sequence, are typically identified by determining whether the bacteria contains the donor sequence. If the donor sequence is a gene encoding a polypeptide which confers antibiotic resistance on the bacterium, then candidate bacteria can be grown in the presence of the antibiotic. Only bacteria which have successfully incorporated the donor sequence will be able to grow in the presence of the antibiotic. Alternatively, if the donor sequence is a gene encoding a fluorescent polypeptide, for example green fluorescent protein, fluorescence may be used to indicate the presence of the donor sequence.

The invention thus provides a bacterium which is capable of expressing the λ genes exo or a functional equivalent thereof and bet or a functional equivalent thereof, but not the λ gene gam or a functional equivalent thereof and which comprises a donor sequence. In addition, the invention provides a bacterium which is capable of expressing the λ gene bet or a functional equivalent thereof, but not the λ genes exo or a functional equivalent thereof and gam or a functional equivalent thereof and which comprises a donor sequence. If the donor sequence replaces a gene, the bacterium will comprise a mutation in that gene.

Several rounds of allelic exchange according to the invention may be carried out sequentially, each time using a different construct. Alternatively, more than one, for example two, three, four or five or more, constructs for use in the invention can be transferred simultaneously into a bacterium. In this case typically the constructs all have different (a) and (b) sequences. That is, each construct has (a) and (b) sequences specific for a different target sequence. Thus, a bacterium of the invention may comprise mutations in more than one, for example two, three, four, five or more, genes.

After successful recombination, it may be necessary to eliminate the donor sequence from the bacterium. This may be required if the recombinant bacterium is to be used in or as a vaccine. For example, the use or antibiotic resistance genes in live attenuated bacteria is not generally permitted by regulatory authorities.

A construct of the invention may therefore also comprise sequences which may be used to eliminate the donor sequence. Such sequences will typically flank the donor sequence and will generally be positioned between sequence (a) and the donor sequence and sequence (b) and the donor sequence. For example, the donor sequence may be flanked by directly repeated FRT (FLP recognition target) sites. After selection of a recombinant comprising the donor sequence, that donor sequence may be eliminated by using a helper plasmid expressing the FLP recombinase, which acts directly on the directly repeated FRT sites. Alternatively, a recombinase protein may be directly transferred, for example by electroporation, into the cell.

Constructs suitable for use this embodiment of the invention may be constructed by any method known to those skilled in the art. Thus, primers comprising sequence (a) or (b), the FRT sequence (or other sequence to be used for elimination of the donor sequence) and part of the donor sequence could be used in PCR with a donor sequence template. Alternatively, the template may itself comprise the donor sequence flanked by appropriate sequences for elimination to occur. That template can be used in PCR with appropriate primers.

If a particular target sequence cannot be replaced by a donor sequence (i.e., no recombinant can be identified for that sequence), that is a strong indication that the target sequence comprises all or part of an essential gene. An essential gene is a gene which, when missing (eg. because of a chromosomal deletion) or mutated to render it non-functional, results in a lethal phenotype. That is, a gene without which a bacterium cannot survive.

The use of bioinformatics may allow the rapid isolation of further essential genes, i.e. corresponding genes from other bacterial species. A gene identified from a particular species identified in the allelic exchange technique of the invention may be used to search databases containing sequence information from other species, in order to identify orthologous genes from those species. Genes so identified can be tested for being essential by using the allelic exchange technique of the invention. For example, an E. coli gene is identified as essential using a method as described above. This may allow the identification of a putative orthologue from Salmonella. That Salmonella gene may be tested by allelic exchange and the construction of conditional mutants in Salmonella as described above. Further orthologues may be identified in more distantly related organisms.

Suitable bioinformatics programs are well known to those skilled in the art. For example, the Basic Local Alignment Search Tool (BLAST) program (Altschul et al., 1990, J. Mol. Biol. 215, 403-410. and Altschul et al., 1997, Nucl. Acids Res. 25 3389-3402.) may be used. Suitable databases for searching are for example, EMBL, GENBANK, TIGR, EBI, SWISS-PROT and trEMBL.

The invention may be carried out using any bacterium. For example, the bacterium may be a Gram-positive or a Gram-negative bacterium. Preferred bacteria are pathogenic bacteria. The bacterium may be pathogenic for a human or an animal or for a plant.

The bacteria may be for example, from the genera Escherichia, Salmonella, Vibrio, Haemophilus, Neisseria, Yersinia, Bordetella, Brucella,. Shigella, Klebsiella, Enterobacter, Serracia, Proteus, Vibrio, Aeromonas, Pseudomonas, Acinetobacter, Moraxella, Flavobacterium, Actinobacillus, Staphylococcus, Streptococcus, Mycobacterium, Listeria, Clostridium, Pasteurella, Helicobacter, Campylobacter, Lawsonia, Mycoplasma, Bacillus, Agrpbacterium, Rhizobium, Erwinia or Xanthomonas.

Examples of some of the above mentioned genera are Escherichia coli—a cause of diarrhoea in humans; Salmonella typhimurium—the cause of salmonellosis in several animal species; Salmonella typhi—the cause of human typhoid fever; Salmonella enteritidis—a cause of food poisoning in humans; Salmonella choleraesuis—a cause of salmonellosis in pigs; Salmonella dublin—a cause of both a systemic and diarrhoeal disease in cattle, especially of new-born calves; Haemophilus influenzae—a cause of meningitis; Neisseria gonorrhoeae—a cause of gonorrhoea; Yersinia enterocolitica—the cause of a spectrum of diseases in humans ranging from gastroenteritis to fatal septicemic disease; Bordetella pertussis—the cause of whooping cough; Brucella abortus—a cause of abortion and infertility in cattle and a condition known as undulant fever in humans; Vibrio cholerae—a cause of cholera; Clostridium tetani—a cause of tetanus; Bacillus anthracis—a cause of anthrax.

Preferred bacteria are those for which the entire genome has been sequenced and therefore for which it may be possible to target all open reading frames for allelic exchange.

Essential genes of a bacterium identified by the allelic exchange method of the invention and the polypeptides which they encode represent targets for antibacterial substances. Such antibacterials may have human, animal and plant applications.

Furthermore, if a particular gene is essential for a number of different bacteria, that gene and the polypeptide it encodes may represent a target for the identification of a substance with a broad-spectrum of activity.

An essential gene identified by a method described above and the polypeptide which it encodes may be used in a method for identifying an inhibitor of transcription and/or translation of the gene and/or activity of the polypeptide encoded by the gene. Such a substance may be referred to as an inhibitor of an essential gene. Thus, an inhibitor of an essential or conditional essential gene is a substance which, for example, inhibits expression and/or translation of that essential gene and/or activity of the polypeptide encoded by that essential or conditional essential gene (i.e. may inhibit the activity of the gene product).

Any suitable assay may be carried out to determine whether a test substance is an inhibitor of an essential gene. For example, the promoter of an essential gene may be linked to a coding sequence for a reporter polypeptide. Such a construct may be contacted with a test substance under conditions in which, in the absence of the test substance expression of the reporter polypeptide would occur. This would allow the effect of the test substance on expression of the essential gene to be determined.

Substances which inhibit translation of an essential or conditional essential gene may be isolated, for example, by contacting the mRNA of the essential gene with a test substance under conditions that would permit translation of the mRNA in the absence of the test substance. This would allow the effect of the test substance on translation of the essential gene to be determined.

Substances which inhibit activity of a polypeptide encoded by the essential gene may be isolated, for example, by contacting the polypeptide with a substrate for the polypeptide and a test substance under conditions that would permit activity of the polypeptide in the absence of the test substance. This would allow the effect of the test substance on activity of the polypeptide encoded by the essential gene to be determined.

Suitable control experiments can be carried out. For example, a putative inhibitor should be tested for its activity against other promoters, mRNAs or polypeptides to discount the possibility that it is a general inhibitor of gene transcription, translation or polypeptide activity.

Suitable test, substances for inhibitors of essential genes include combinatorial libraries, defined chemical entities, peptides and peptide mimetics, oligonucleotides and natural product libraries. The test substances may be used in an initial screen of, for example, ten substances per reaction, and the substances of batches which show inhibition tested individually. Furthermore, antibody products (for example, monoclonal and polyclonal antibodies, single chain antibodies, chimaeric antibodies and CDR-grafted antibodies) may be used.

An inhibitor of an essential gene is a substance which inhibits expression and/or translation of that essential gene and/or activity of the polypeptide encoded by that essential gene. Preferred substances are those which inhibit essential gene expression and/or translation and/or activity by at least 10%, at least 20%, at least 30%, at least 40%, at least 50%, at least 60%, at least 70%, at least 80%, at least 90%, at least 95% or at least 99% at a concentration of the inhibitor of 1 μgml⁻¹, 10 μgml⁻¹, 100 μgml⁻¹, 500 μgml⁻¹, 1 mgml⁻¹, 10 mgml⁻¹, 100 mg ml⁻¹. The percentage inhibition represents the percentage decrease in expression and/or translation and/or activity in a comparison of assays in the presence and absence of the test substance. Any combination of the above mentioned degrees of percentage inhibition and concentration of inhibitor may be used to define an inhibitor of the invention, with greater inhibition at lower concentrations being preferred.

Test substances which show activity in assays such as those described above can be tested in in vivo systems, such as an animal model of infection for or a plant model to test for pesticidal activity. Thus, candidate inhibitors could be tested for their ability to attenuate bacterial infections in mice or for their ability to inhibit growth of plants in the case of a pesticide.

Inhibitors of bacterial essential genes may be used in a method of treatment of the human or animal body by therapy. In particular such substances may be used in a method of treatment of a bacterial infection. Such substances may also be used for the manufacture of a medicament for use in the treatment of a bacterial infection. The condition of a patient suffering from such a bacterial infection can be improved by administration of an inhibitor. A method of treating a host suffering from a bacterial infection may comprise administering to the host an effective amount of an inhibitor of the invention. The host may be a mammalian host, typically human but may be an animal. A therapeutically effective amount of an inhibitor may be given to a human patient in need thereof.

Inhibitors of bacterial essential gene may be administered in a variety of dosage forms. Thus, they can be administered orally, for example as tablets, troches, lozenges, aqueous or oily suspensions, dispersible powders or granules. The inhibitors may also be administered parenterally, either subcutaneously, intravenously, intramuscularly, intrasternally, transdermally or by infusion techniques. The inhibitors may also be administered as suppositories. A physician will be able to determine the required route of administration for each particular patient.

The formulation of an inhibitor for use in preventing or treating a bacterial or fungal infection will depend upon factors such as the nature of the exact inhibitor, whether a pharmaceutical or veterinary use is intended, etc. An inhibitor may be formulated for simultaneous, separate or sequential use.

An inhibitor is typically formulated for administration in the present invention with a pharmaceutically acceptable carrier or diluent, that is the inhibitor may be formulated as part of a pharmaceutical composition. The pharmaceutical carrier or diluent may be, for example, an isotonic solution. For example, solid oral forms may contain, together with the active compound, diluents, e.g. lactose, dextrose, saccharose, cellulose, corn starch or potato starch; lubricants, e.g. silica, talc, stearic acid, magnesium or calcium stearlate, and/or polyethylene glycols; binding agents; e.g. starches, gum arabic, gelatin, methylcellulose, carboxymethylcellulose or polyvinyl pyrrolidone; disaggregating agents, e.g. starch, alginic acid, alginates or sodium starch glycolate; effervescing mixtures; dyestuffs; sweeteners; wetting agents, such as lecithin, polysorbates, laurylsulphates; and, in general, non-toxic and pharmacologically inactive substances used in pharmaceutical formulations. Such pharmaceutical preparations may be manufactured in a known manner, for example, by means of mixing, granulating, tabletting, sugar-coating, or film-coating processes.

Liquid dispersions for oral administration may be syrups, emulsions or suspensions. The syrups may contain as carriers, for example, saccharose or saccharose with glycerine and/or mannitol and/or sorbitol.

Suspensions and emulsions may contain as carrier, for example a natural gum, agar, sodium alginate, pectin, methylcellulose, carboxymethyicellulose, or polyvinyl alcohol. The suspensions or solutions for intramuscular injections may contain, together with the active compound, a pharmaceutically acceptable carrier, e.g. sterile water, olive oil, ethyl oleate, glycols, e.g. propylene glycol, and if desired, a suitable amount of lidocaine hydrochloride.

Solutions for intravenous administration or infusion may contain as carrier, for example, sterile water or preferably they may be in the form of sterile, aqueous, isotonic saline solutions.

A therapeutically effective amount of an inhibitor is administered to a patient. The dose of an inhibitor may be determined according to various parameters, especially according to the substance used; the age, weight and condition of the patient to be treated; the route of administration; and the required regimen. Again, a physician will be able to determine the required route of administration and dosage for any particular patient. A typical daily dose is from about 0.1 to 50 mg per kg of body weight, according to the activity of the specific inhibitor, the age, weight and conditions of the subject to be treated, the type and severity of the degeneration and the frequency and route of administration. Preferably, daily dosage levels are from 5 mg to 2 g.

Inhibitors of bacterial essential genes may be administered to plants in order to prevent or treat bacterial infections. Thus inhibitors of the invention may be useful as bacteriocides/pesticides.

The inhibitors of the present invention ate normally applied in the form of compositions together with one or more agriculturally acceptable carriers or diluents and can be applied to the crop area or plant to be treated, simultaneously or in succession with further compounds.

The inhibitors of the invention can be selective bacteriocides/pesticides or mixtures of several of these preparations, if desired together with further carriers, surfactants or application-promoting adjuvants customarily employed in the art of formulation. Suitable carriers and diluents correspond to substances ordinarily employed in formulation technology, e.g. natural or regenerated mineral substances, solvents, dispersants, wetting agents, tackifiers, binders or fertilizers.

A preferred method of applying inhibitors of the present invention or an agrochemical composition which contains at least one of the inhibitors is leaf application. The number of applications and the rate of application depend on the intensity of infestation by the pathogen. However, the inhibitors can also penetrate the plant through the roots via the soil (systemic action) by impregnating the locus of the plant with a liquid composition, or by applying the compounds in solid form to the soil, e.g. in granular form (soil application). The inhibitors may also be applied to seeds (coating) by impregnating the seeds either with a liquid formulation containing active ingredients, or coating them with a solid formulation. In special cases, further types of application are also possible, for example, selective treatment of the plant stems or buds.

The inhibitors are used in unmodified form or, preferably, together with the adjuvants conventionally employed in the art of formulation, and are therefore formulated in known manner to emulsifiable concentrates, coatable pastes, directly sprayable or dilutable solutions, dilute emulsions, wettable powders, soluble powders, dusts, granulates, and also encapsulations, for example, in polymer substances. Like the nature of the compositions, the methods of application, such as spraying, atomizing, dusting, scattering or pouring, are chosen in accordance with the intended objectives and the prevailing circumstances. Advantageous rates of application are normally from 50 g to 5 kg of active ingredient (a.i.) per hectare (“ha”, approximately 2.471 acres), preferably from 100 g to 2 kg a.i./ha, most preferably from 200 g to 500 g a.i./ha.

The formulations, compositions or preparations containing the inhibitors and, where appropriate, a solid or liquid adjuvant, are prepared in known manner, for example by homogeneously mixing and/or grinding inhibitors with extenders, for example solvents, solid carriers and, where appropriate, surface-active compounds (surfactants).

Suitable solvents include aromatic hydrocarbons, preferably the fractions having 8 to 12 carbon atoms, for example, xylene mixtures or substituted naphthalenes, phthalates such as dibutyl phthalate or dioctyl phthalate, aliphatic hydrocarbons such as cyclohexane or paraffins, alcohols and glycols and their ethers and esters, such as ethanol, ethylene glycol, monomethyl or monoethyl ether, ketones such as cyclohexanone, strongly polar solvents such as N-methyl-2-pyrrolidone, dimethyl sulfoxide or dimethyl formamide, as well as epoxidized vegetable oils such as epoxidized coconut oil or soybean oil; or water.

The solid carriers used e.g. for dusts and dispersible powders, are normally natural mineral fillers such as calcite, talcum, kaolin, montinorillonite or attapulgite. In order to improve the physical properties it is also possible to add highly dispersed silicic acid or highly dispersed absorbent polymers. Suitable granulated adsorptive carriers are porous types, for example pumice, broken brick, sepiolite or bentonite; and suitable nonsorbent carriers are materials such as calcite or sand. In addition, a great number of pregranulated materials of inorganic or organic nature can be used, e.g. especially dolomite or pulverized plant residues.

Depending on the nature of the inhibitor to be used in the formulation, suitable surface-active compounds are nonionic, cationic and/or anionic surfactants having good emulsifying, dispersing and wetting properties. The term “surfactants” will also be understood as comprising mixtures of surfactants.

Suitable anionic surfactants can be both water-soluble soaps and water-soluble synthetic surface-active compounds.

Suitable soaps are the alkali metal salts, alkaline earth metal salts or unsubstituted or substituted ammonium salts of higher fatty acids (chains of 10 to 22 carbon atoms), for example the sodium or potassium salts of oleic or stearic acid, or of natural fatty acid mixtures which can be obtained for example from coconut oil or tallow oil. The fatty acid methyltaurin salts may also be used.

More frequently, however, so-called synthetic surfactants are used, especially fatty sulfonates, fatty sulfates, sulfonated benzimidazole derivatives or alkylarylsulfonates.

The fatty sulfonates or sulfates are usually in the form of alkali metal salts; alkaline earth metal salts or unsubstituted or substituted ammoniums salts and have a 8 to 22 carbon alkyl radical which also includes the alkyl moiety of alkyl radicals, for example, the sodium or calcium salt of lignonsulfonic acid, of dodecylsulfate or of a mixture of fatty alcohol sulfates obtained from natural fatty acids. These compounds also comprise the salts of sulfuiric acid esters and sulfonic acids of fatty alcohol/ethylene oxide adducts. The sulfonated benzimidazole derivatives preferably contain 2 sulfonic acid groups and one fatty acid radical containing 8 to 22 carbon atoms. Examples of alkylarylsulfonates are the sodium, calcium or triethanolamine salts of dodecylbenzenesulfonic acid, dibutylnaphthalenesulfonic acid, or of a naphthalenesulfonic acid/formaldehyde condensation product. Also suitable are corresponding phosphates, e.g. salts of the phosphoric acid ester of an adduct of p-nonylphenol with 4 to 14 moles of ethylene oxide.

Non-ionic surfactants are preferably polyglycol ether derivatives of aliphatic or cycloaliphatic alcohols, or saturated or unsaturated fatty acids and alkylphenols, said derivatives containing 3 to 30 glycol ether groups and 8 to 20 carbon atoms in the (aliphatic) hydrocarbon moiety and 6 to 18 carbon atoms in the alkyl moiety of the alkylphenols.

Further suitable non-ionic surfactants are the water-soluble adducts of polyethylene oxide with polypropylene glycol, ethylenediamine propylene glycol and alkylpolypropylene glycol containing 1 to 10 carbon atoms in the alkyl chain, which adducts contain 20 to 250 ethylene glycol ether groups and 10 to 100 propylene glycol ether groups. These compounds usually contain 1 to 5 ethylene glycol units per propylene glycol unit.

Representative examples of non-ionic surfactants are nonylphenolpolyethoxyethanols, castor oil polyglycol ethers, polypropylene/polyethylene oxide adducts, tributylphenoxypolyethoxyethanol, polyethylene glycol and octylphenoxyethoxyethanol. Fatty acid esters of polyoxyethylene sorbitan and polyoxyethylene sorbitan trioleate are also suitable non-ionic surfactants.

Cationic surfactants are preferably quaternary ammonium salts which have, as N-substituent, at least one C₈-C₂₂ alkyl radical and, as further substituents, lower unsubstituted or halogenated alkyl, benzyl or lower hydroxyalkyl radicals. The salts are preferably in the form of halides, methylsulfates or ethylsulfates, e.g. stearyltrimethylammonium chloride or benzyldi(2-chloroethyl)ethylammonium bromide.

The surfactants customarily employed in the art of formulation are described, for example, in “McCutcheon's Detergents and Emulsifiers Annual”, MC Publishing Corp. Ringwood, N.J., 1979, and Sisely and Wood, “Encyclopaedia of Surface Active Agents,” Chemical Publishing Co., Inc. New York, 1980.

The agrochemical compositions usually contain from about 0.1 to about 99% preferably about 0.1 to about 95%, and most preferably from about 3 to about 90% of the active ingredient, from about 1 to about 99.9%, preferably from about 1 to 99%, and most preferably from about 5 to about 95% of a solid or liquid adjuvant, and from about 0 to about 25%, preferably about 0.1 to about 25%, and most preferably from about 0.1 to about 20% of a surfactant.

Whereas commercial products are preferably formulated as concentrates, the end user will normally employ dilute formulations.

The allelic exchange technique of the invention may be used to prepare attenuated live vaccines. The principle behind vaccination is to induce an immune response in the host thus providing protection against subsequent challenge with a pathogen. This may be achieved by inoculation with a live attenuated strain of the pathogen, i.e. a strain having reduced virulence such that it does not cause the disease caused by the virulent pathogen. Typically, attenuation is achieved by mutating genes which are required for virulence/pathogenicity or viability in a host.

The allelic exchange method of the invention may be used to introduce mutations into a bacterium which result in attenuation of that bacterium. The attenuated bacterium can then be used in a vaccine. Thus, the invention provides an attenuated bacterium which is capable of expressing: (i) the λ genes exo or a functional equivalent thereof and bet or a functional equivalent thereof, but not the λ gene gam or a functional equivalent thereof; or (ii) the λ gene bet or a functional equivalent thereof, but not the λ genes exo or a functional equivalent thereof and gam or a functional equivalent thereof, and which is attenuated by a non-reverting mutation in at least one gene, for example two, three, four, five or more, which is required for pathogenicity.

The mutations introduced into a bacterium for use in a vaccine generally knock-out the function of the gene completely. This may be achieved either by abolishing synthesis of any polypeptide at all from the gene or by making a mutation that results in synthesis of non-functional polypeptide. In order to abolish synthesis of polypeptide, either the entire gene or its 5′-end may be replaced using the allelic exchange technique of the invention. A deletion or insertion, again using the allelic exchange method of the invention, within the coding sequence of a gene may be used to create a gene that synthesises only non-functional polypeptide (e.g. polypeptide that contains only the N-terminal sequence of the wild-type protein).

The bacterium may have mutations in one or more, for example two, three or four genes. The mutations are non-reverting mutations. These are mutations that show essentially no reversion back to the wild-type when the bacterium is used as a vaccine. Such mutations are typically insertions and deletions. Insertions and deletions are preferably large, typically at least 10 nucleotides in length, for example from 10 to 600 nucleotides. Preferably, the whole coding sequence is deleted. Deletions may be carried out by replacing a target coding sequence with a donor sequence and then subsequently removing the donor sequence as is described above.

The bacterium used in the vaccine preferably contains only defined mutations, i.e. mutations which are characterised. It is clearly undesirable to use a bacterium which has uncharacterised mutations in its genome as a vaccine because there would be a risk that the uncharacterised mutations may confer properties on the bacterium that cause undesirable side-effects.

In addition, if the bacterium is to be used in a vaccine and the exo and bet genes or bet gene alone are expressed from a plasmid, it is preferable to remove the plasmid before the bacterium is used in vaccination. There a number of ways of removing plasmids, which are well known to those skilled in the art.

For example, the plasmid expressing exo and bet, or bet alone, may be temperature sensitive (ts). Thus, a ts-replicon may be incorporated into the plasmid. The temperature-sensitive pSC101 replicon is functional at 30° C., but not at 43° C.; (Hamilton et-al., 1989, J. Bacteriology 171, 4617-4622). The use of plasmids based on this replicon allows allelic exchange to be carried out in cultures grown at 30° C. Following allelic exchange, the growth temperature of the E.coli host can be raised to 43° C. Under these conditions, the replicon cannot function, and consequently colonies can be isolated that are plasmid-free.

As an alternative, the loci-disrupted may be transduced into a “clean” genetic background, using a transducing phage. A typical bacteriophage used for generalized transduction in E.coli is P1. When this phage infects E.coli, it can grow either as a lysogen—followed by subsequent lytic growth—or it can enter a lytic growth cycle right away. When the phage is in the lytic growth cycle, it replicates its own DNA chromosome as long linear concatemers (i.e., one genome length after another on a single molecule of DNA). These linear fragments are processed into genome lengths for packaging into phage capsids by specific endonucleases. A bi-product of these processing events is the packaging of host E.coli chromosomal DNA into some of the capsids.

This packaging process can be used for the transfer of markers from one E.coli host to another, ie the marker can be ‘transduced’ to a clean genetic background. For example, following P1 infection of a donor host E. coli that has undergone allelic exchange, packaged phage particles can be purified. These phage can be mixed with a recipient E.coli host, and if the DNA in the capsid is bacterial in origin, no infection will result. However, the bacterial DNA will be injected into the recipient E.coli, and recombine with the host genome. If the bacterial DNA from the donor strain contains a drug-resistance marker (eg a kanamycin cassette), then antibiotic selection can be used to isolate mutants that possess the new phenotype.

Particular genes which may be targeted for allelic exchange using a method of the invention for attenuating a bacterium include aroA, aroC, aroD and aroE), pur, htrA, ompR, ompF, ompC, galE, cya, crp and phoP and any combination thereof. Preferred combinations include aroA/purA and aroA/aroC double mutants.

The attenuated bacterium of the invention may be genetically engineered to express an antigen that is not expressed by the native bacterium (a “heterologous antigen”), so that the attenuated bacterium acts as a carrier of the heterologous antigen. The antigen may be from another organism, so that the vaccine provides protection against the other organism. A multivalent vaccine may be produced which not only provides immunity against the virulent parent of the attenuated bacterium but also provides immunity against the other organism. Furthermore, the attenuated bacterium may be engineered to express more than one heterologous antigen, in which case the heterologous antigens may be from the same or different organisms.

The heterologous antigen may be a complete protein or a part of a protein containing an epitope. The antigen may be from a virus, prokaryote or a eukaryote, for example another bacterium, a yeast, a fungus or a eukaryotic parasite. The antigen may be from an extracellular or intracellular protein. More especially, the antigenic sequence may be from E.coli, tetanus, hepatitis A, B or C virus, human rhinovirus such as type 2 or type 14, herpes simplex virus, poliovirus type 2 or 3, foot-and-mouth disease virus, influenza virus, coxsackie virus or Chlamydia trachomatis. Useful antigens include non-toxic components of E.coli heat labile toxin, E.coli K88 antigens, ETEC colonization factor antigens, P.69 protein from B.pertussis and tetanus toxin fragment C.

The DNA encoding the heterologous antigen is expressed from a promoter that is active in vivo. Two promoters that have been shown to work well in Salmonella are the nirB promoter and the htrA promoter. For expression of the ETEC colonization factor antigens, the wild-type promoters could be used.

A DNA construct comprising the promoter operably linked to DNA encoding the heterologous antigen may be made and transformed into the attenuated bacterium using conventional techniques. Transformants containing the DNA construct may be selected, for example by screening for a selectable marker on the construct. Bacteria containing the construct may be grown in vitro before being formulated for administration to the host for vaccination purposes.

The vaccine may be formulated using known techniques for formulating attenuated bacterial vaccines. The vaccine is advantageously presented for oral administration, for example in a lyophilised encapsulated form. Such capsules may be provided with an enteric coating comprising, for example, Eudragate “S” (Trade Mark), Eudragate “L” (Trade Mark), cellulose acetate, cellulose phthalate or hydroxypropylmethyl cellulose. These capsules may be used as such, or alternatively, the lyophilised material may be reconstituted prior to administration, e.g. as a suspension. Reconstitution is advantageously effected in a buffer at a suitable pH to ensure the viability of the bacteria. In order to protect the attenuated bacteria and the vaccine from gastric acidity, a sodium bicarbonate preparation is advantageously administered before each administration of the vaccine. Alternatively, the vaccine may be prepared for parenteral administration, intranasal administration or intramuscular administration.

The vaccine may be used in the vaccination of a mammalian host, particularly a human host but also an animal host. An infection caused by a microorganism, especially a pathogen, may therefore be prevented by administering an effective dose of a vaccine prepared according to the invention. The dosage employed will ultimately be at the discretion of the physician, but will be dependent on various factors including the size and weight of the host and the type of vaccine formulated. However, a dosage comprising the oral administration of from 10⁷ to 10¹¹ bacteria per dose may be convenient for a 70 kg adult human host.

The following Example illustrates the invention:

### EXAMPLE

### Materials and Methods

Unless indicated otherwise, the methods used are standard biochemical techniques. Examples of suitable general methodology textbooks include Sambrook et al., Molecular Cloning, a Laboratory Manual (1989) and Ausubel et al., Current Protocols in Molecular Biology (1995), John Wiley & Sons, Inc.

Previous studies have shown that three genes, encoding exo, bet and gam are the minimal set required to generate PCR-derived allelic exchange with λ gene products. In order to determine whether all three of the exo, bet and gam gene products are required for the procedure, we have tested expression cassettes generating: (i) the exo and bet proteins; (ii) the bet protein alone; and (iii) the exo gene alone, under the control of an arabinose inducible promoter. For control experiments, the three genes exo, bet and gam were tested in a single cassette.

The following combinations of primers were used:

(a) The exo and bet genes were cloned into pBAD using:

| | |
|---|---|
| bet forward primer | |
| (NcoI site in bold) | |
| 5′ gaggtataaaacccatgggtactgcactcg 3′ | (SEQ ID NO: 1) |
| exo reverse primer | |
| (HindIII site in bold) | |
| 5′ tatcgtgaggaagcttcatcgccattg 3′ | (SEQ ID NO: 2) |

(b) The exo gene alone was cloned into pBAD using:

exo reverse primer (SEQ ID NO: 2)

| | |
|---|---|
| exo forward primer (NcoI site in bold): | |
| 5′ gaaggtggcaccatggcaccggacatta 3 | (SEQ NO: 3) |

(c) The bet gene alone was cloned into pBAD using:

bet forward primer (SEQ ID NO: 1)

| | |
|---|---|
| bet reverse primer | |
| (EcoRI site in bold): | |
| 5′ ggataatgtccgaattcatgctgccac 3′ | (SEQ NO: 4) |

(d) The exo, bet and gam genes were cloned into pBAD using:

exo reverse primer (SEQ ID NO: 1)

| | |
|---|---|
| gam forward primer | |
| (NcoI site in bold): | |
| 5′ gacgcttataaaccatggatattaatactg 3′ | (SEQ ID NO: 5) |

In order to determine if the exo and bet gene products can direct allelic exchange, four strains of E.coli MG1665 expressing either (i) pBAD exo, bet and gain; (ii) pBAD exo; (iii) pBAD bet; or (iv) pBAD exo and bet were grown up in LB at 37° C. When each reached OD₆₀₀ 0.25, the cells were induced with arabinose (final concentration 0.2% (w/v)) and grown to OD₆₀₀ 0.5. After 15 min on ice, 3 ml aliquots of cells were pelleted for each strain and then electrocompetent cells were prepared by washing the cells with 2×1 ml ice cold water followed by washing with 1 ml ice cold 10% glycerol. The cells were resuspended in 70 μl 10% glycerol and stored at −80° C. until needed. DNA samples were added in a volume of 10 μl.

In order to test for allelic exchange, the ilvA gene (a gene encoding threonine deaminase, the first step of isoleucine biosynthesis) was chosen. Each experiment included (i) a negative control of no added DNA; (ii) a positive control of the pUC4K plasmid (Pharmacia) and (iii) 0.5 μg of kan cassette ilvA arms. The ilvA composite oligonucleotides were as set out below. These oligonucleotides contain a region of identity with the left- and right-arms of ilvA, and a region of identity with the kanamycin resistance cassette. PCR was carried out on a kan-resistance cassette.

The kan-resistance plasmid for these allelic exchange experiments was generated by using PCR to amplify the resistance determinant from pUC4K (Pharmacia). The oligonucleotides used were:

| | |
|---|---|
| 5′-gcgctgaggTCTAGAtcgtgaagaagg-3′ | (SEQ ID NO: 6) |
| 5′-acacaggaaacaTCTAGAaccatgatta-3′ | (SEQ ID NO: 7) |
| (XbaI site shown underlined) | |

Following PCR, (94° C., 1 min; 55° C., 1 min; 72° C., 2 min, for 25 cycles) an XbaI site was introduced into the kanamycin resistance fragment. Following digestion with XbaI and gel purification, the fragment was cloned into the XbaI site of pUC18. The resulting plasmid was purified, and the Xbal-kan fragment gel purified, and used as a substrate for the generation of linear allelic exchange fragments.

Oligonucleotides with gene-specific sequences were used to amplify the purified kan cassette using the following PCR conditions: 94° C., 1 min; 65° C., 1 min; 72° C., 2 min, for 30 cycles.

| | |
|---|---|
| ilvA foward primer | |
| 5′cctggcaaccagcgccgacaaaggcgcggtgcgcgataaatcgaaactggggggttaatagccgccgtcccgtcaagtcagcg3′ | (SEQ NO: 8) |
| ilvA reverse primer | |
| 5′cacaaatgacgttgtcgcgcgggtaggcctgataagcgaagcgctatcaggcatttttccccccggatccgtcgacctgcag3′ | (SEQ NO: 9) |

Following amplification, PCR products were purified using a Quiaquick nucleotide removal kit (Qiagen). Lamda exo functions as an exonuclease to generate single-stranded DNA ends from a double-stranded template. In order to determine if the exo gene product protein was necessary for successful allelic exchange, experiments were carried out with both double-stranded and single-stranded template. The single-stranded template was generated by heating PCR-generated double-stranded template at 94° C. for 2 min and immediately quenching on ice for 2 min.

The single-stranded and double-stranded templates were electroporated into E.coli MG1665 expressing the different combinations of λ-genes described above. Immediately following electroporation (using a BioRad E.coli pulser 2.5 kv), 500 μl of L-broth were added to the electroporation cuvette, and the cells shaken at 37° C. for 90 min. Cells were plated on L-agar plates (containing 25 μg /ml kanamycin) and grown overnight at 37° C.

Colonies obtained the next day were picked into 20 μl PCR mix and the correct insertion assessed with a primer pair based on the ilvA gene and the kanamycin cassette.

| | | |
|---|---|---|
| ilvA test primer | 5′-gaaaaatcgtgaacgtcaggtctcc-3′ | (SEQ ID NO: 10) |
| kan test primer | 5′-attcctgtttgtaattgtcc-3′ | (SEQ ID NO: 11) |

PCR conditions (carried out in Promega Taq buffer and dNTPs) for the test reaction were 94° C., 1 min; 55° C., 1 min; 72° C., 2 min for a total of 25 cycles. For a correct insertion event, the expected PCR product is 590 bp.

The results- are shown overleaf:

### Results

Experiment 1 (electroporation with 0.7 μg linear ilvA PCR product)
Experiment run in duplicate

| | |
|---|---|
| ds-DNA | ss-DNA |

| | | | | |
|---|---|---|---|---|
| pBAD exo, bet and gam, | 21 | 95 | 10 | 18 |
| pBAD exo and bet | 33 | 11 | 33 | 37 |
| pBAD exo alone | 3 | 0 | 29 | 14 |
| pBAD bet alone | 0 | 0 | 0 | 0 |
| pBAD-negative control | 0 | 0 | 0 | 0 |

Experiment 2a (electroporation with a range of concentrations of linear
ilvA PCR products in cells expressing bet) Experiment run in duplicate

| | |
|---|---|
| ds-DNA | ss-DNA |

| | | | | |
|---|---|---|---|---|
| 1.00 μg | 0 | 0 | 11 | 14 |
| 0.75 μg | 0 | 0 | 3 | 2 |
| 0.50 μg | 0 | 2 | 0 | 0 |
| 0.25 μg | 0 | 1 | 0 | 0 |
| 0.10 μg | 1 | 0 | 0 | 0 |

Experiment 2b (electroporation with a range of concentrations of linear
ilvA PCR products in cells expressing exo, bet and gam) Experiment
run in duplicate

| | |
|---|---|
| ds-DNA | ss-DNA |

| | | | | |
|---|---|---|---|---|
| 1.00 μg | 54 | 33 | 2 | 1 |
| 0.75 μg | 26 | 9 | 0 | 1 |
| 0.50 μg | 42 | 6 | 0 | 0 |
| 0.25 μg | 55 | 2 | 0 | 0 |
| 0.10 μg | 35 | 57 | 0 | 0 |

### Conclusion

Previous reports have suggested that in order for allelic exchange to function with the λ recombination genes, the three components exo, bet and gam, are required. We show here that the presence of the Exo and Bet proteins in an E.coli cell can allow the generation of allelic exchange mutants at a similar frequency to exo, bet and gain expressing cells. Importantly, the expression of Bet alone is sufficient to generate allelic exchange mutants, particularly when single-stranded DNA is used as a template.

1-31. (Cancel).

32. A method for replacing a target sequence of a bacterial chromosome with a donor sequence, which method comprises:
(a) providing a bacterium which is capable of expressing:
(1) the λ genes exo or a functional equivalent thereof and bet or a functional equivalent thereof, but not the λ gene gam or a functional equivalent thereof; or
(2) the λ gene bet or a functional equivalent thereof, but not the λ genes exo or a functional equivalent thereof and gam or a functional equivalent thereof;
(b) providing a polynucleotide construct which comprises:
(i) a sequence which corresponds to a first sequence flanking the left hand side of the target sequence;
(ii) a second sequence corresponding to a sequence flanking the right hand side of the target sequence; and
(iii) positioned between (i) and (ii), the donor sequence; and
(c) introducing the polynucleotide construct into the bacterium, thereby to replace the target nucleotide sequence with the sequence different from that of the target nucleotide sequence.

33. A method according to claim 32, which further comprises the step:
(d) identifying whether the bacterium has incorporated the donor sequence.

34. A method according to claim 32, wherein the donor sequence is different from the target sequence.

35. A method according to claim 32, wherein the polynucleotide construct is double-stranded DNA or single-stranded DNA.

36. A method according to claim 32, wherein the λ genes exo or a functional equivalent thereof and bet or a functional equivalent thereof or the λ gene bet or a functional equivalent thereof are expressed from a plasmid or a λ prophage.

37. A method according to claim 32, wherein the λ genes exo or a functional equivalent thereof and bet or a functional equivalent thereof or the λ gene bet or a functional equivalent thereof are expressed under the control of an arabinose inducible promoter.

38. A method according to claim 32, wherein the donor sequence comprises sequence encoding a polypeptide which confers antibiotic resistance on a bacterium in which that polypeptide is expressed.

39. A method according to claim 32, wherein the bacterium is a Gram-negative or a Gram-positive bacterium.

40. A method according to claim 39, wherein the bacterium is from the genus Escherichia, Salmonella, Vibrio, Haemophilus, Neisseria, Yersinia, Bordetella, Brucella, Shigella, Klebsiella, Enterobacter, Serracia, Proteus, Vibrio, Aeromonas, Pseudomonas, Acinetobacter, Moraxella, Flavobacterium, Actinobacillus, Staphylococcus, Streptococcus, Mycobacterium, Listeria, Clostridium, Pasteurella, Helicobacter, Campylobacter, Lawsonia, Mycoplasma, Bacillus, Agrobacterium, Rhizobium, Erwinia or Xanthomonas.

41. A method according to claim 40, wherein the bacterium is Escherichia coli, Salmonella typhimurium, Salmonella typhi, Salmonella enteritidis, Salmonella choleraesuis, Salmonella dublin, Haemophilus influenzae, Neisseria gonorrhoeae, Yersinia entero-colitica, Bordetella pertussis, Brucella abortus, Vibrio cholerae, Clostridium tetani or Bacillus anthracis.

42. A method according to claim 32, wherein the polynucleotide construct is introduced into the bacterium by electroporation.

43. A method according to claim 32 for the preparation of a modified bacterium.

44. A method according to claim 32, wherein the target sequence comprises all or part of a gene which is required for pathogenicity.

45. A method according to claim 44 for the preparation of an attenuated bacterium.

46. An attenuated bacterium obtained by a method according to claim 45.

47. An attenuated bacterium which is capable of expressing:
(a) the λ genes exo or a functional equivalent thereof and bet or a functional equivalent thereof, but not the λ gene gam or a functional equivalent thereof; or
(b) the λ gene bet or a functional equivalent thereof, but not the λ genes exo or a functional equivalent thereof and gam or a functional equivalent thereof, and which is attenuated by a non-reverting mutation in at least one gene which is required for pathogenicity.

48. A vaccine comprising an attenuated bacterium according to claim 47 and a pharmaceutically acceptable carrier or diluent.

49. A method for raising an immune response in a mammalian host, which comprises administering to the host a bacterium according to claim 47.

50. A method according to claim 32 for identifying an essential gene wherein inability to replace the target sequence is indicative of that target sequence comprising all or part of an essential gene.

51. Use of an essential gene identified by a method according to claim 50, or the polypeptide encoded by a said gene, in a method for identifying an inhibitor of transcription and/or translation of that gene and/or activity of a polypeptide encoded by that gene.

52. A method for identifying: (i) an inhibitor of transcription and/or translation of an essential gene; and/or (ii) an inhibitor of activity of a polypeptide encoded by a said gene, which method comprises:
(a) identifying an essential gene by a method according to claim 50; and
(b) determining whether a test substance can inhibit transcription and/or translation of a said gene and/or activity of a polypeptide encoded by a said gene.

53. An inhibitor identified by a method according to claim 52.

54. A pharmaceutical composition comprising an inhibitor according to claim 53 and a pharmaceutically acceptable carrier or diluent.

55. A method of treating a host suffering from a bacterial infection, which comprises administering to the host an effective amount of an inhibitor according to claim 53.

56. A method for the preparation of a pharmaceutical composition, which method comprises:
(a) identifying: (i) an inhibitor of transcription and/or translation of an essential gene; and/or (ii) an inhibitor of activity of a polypeptide encoded by a said gene by a method according to claim 52; and
(b) formulating the inhibitor identified in step (a) with a pharmaceutically acceptable carrier or diluent.

57. A method of treating a host suffering from a bacterial infection, which method comprises:
(a) identifying: (i) an inhibitor of transcription and/or translation of an essential gene; and/or (ii) an inhibitor of activity of a polypeptide encoded by a said gene by a method according to claim 52;
(b) formulating the inhibitor identified in step (a) with a pharmaceutically acceptable carrier or diluent; and
(c) administering to the host an effective amount of the pharmaceutical composition of step (b).
